(19) United States
(12) Patent Application Publication
(10) Pub. No.: US 2024/0245896 A1
(43) Pub. Date: Jul. 25, 2024

(54) MEDICAL DEVICE WITH A FOUR- LEAD THREAD, LUER CONNECTOR

(71) Applicant: Becton, Dickinson and Company, Franklin Lakes, NJ (US)

(72) Inventor: Paul P. Marici, Piscataway, NJ (US)

(73) Assignee: Becton, Dickinson and Company, Franklin Lakes, NJ (US)

(21) Appl. No.: 18/157,922

(22) Filed: Jan. 23, 2023

(51) Int. Cl. A61M 39/10; A61M 5/31; A61M 25/00

(52) U.S. Cl. CPC A61M 39/10; A61M 5/3134; A61M 25/0097; A61M 2039/1038

(57) ABSTRACT

Medical devices incorporate four-lead thread, male or female Luer connectors, also referred to as four-start thread, Luer connectors. The Luer connector comprises a monolithic, molded-polymer, tubular body having inner and outer wall circumferential surfaces and four-lead helical threads formed on one of the wall circumferential surfaces. The helical threads circumscribe a nested, concentrically aligned, frusto-conical, Luer-tapered, mating surface. The four-lead thread configuration reduces axial, cross-sectional area of each thread, which facilitates more uniform mold filling and cooling while injection molding the medical device, more uniform finished product and material savings, compared with traditional two-lead thread Luer connectors.

### TECHNICAL FIELD

The present disclosure generally relates to medical devices, which incorporate threaded Luer connectors. More particularly, the present disclosure relates to medical devices with male or female Luer connectors having four-lead, also referred to as four-start, helical thread patterns.

### BACKGROUND

Luer connectors are connection collars that are the standard way of attaching medical devices, such as syringes, catheters, hubbed needles, IV tubes, etc. to each other. A Luer connector consists of male and female interlocking tubes, slightly tapered to hold together better with even just a simple pressure/twist fit. Some Luer connector embodiments include an additional outer rim of threading, allowing them to be more securely coupled to mating threaded connectors (sometimes referred to as a locking Luer connector or a Luer lock connector, or simply a Luer lock). The Luer connector male end is typically incorporated within a distal end of a syringe and can interlock and connect to the female end located on the vascular access device (VAD), such as an intravenous catheter or a hub, or to a needle hub. A syringe cap with a female Luer connector is often used to seal a syringe by coupling to the male Luer connector of the syringe, to maintain sterility and prevent fluid leakage. Typically, Luer connectors and associated medical devices are fabricated by injection molding of plastic polymers.

ISO 80369-7:2016 defines a specification for standard Luer connectors including a 6% taper between the distal end and the proximal end. A male standard Luer connector increases from the open distal end to the proximal end. A female standard Luer connector decreases from the open proximal end to the distal end. According to ISO 80369-7:2016, a male standard Luer connector has an outer cross-sectional diameter measured 0.75 mm from the distal end of the tip of between 3.970 mm and 4.072 mm. The length of the male standard Luer taper is between 7.500 mm to 10.500 mm. The outer cross-sectional diameter measured 7.500 mm from the distal end of the tip is between 4.376 mm and 4.476 mm. As used herein, the phrases “male standard Luer connector” and “female standard Luer connector” shall refer to connectors having the dimensions described in ISO 80369-7, which is hereby incorporated by reference in its entirety.

ISO 80369-7 compliant, male or female Luer lock connectors have two-start or two-lead, helical threads with trapezoidal profile, alternating ribs and grooves. The relevant ISO specification for the Luer taper diameter and the outer diameter of the thread ribs requires relatively thick tubular wall construction, which complicates injection molding of the medical device that incorporates the Luer connector. In general, thicker wall construction of an injection-molded, plastic component requires longer injection and cooling time in a mold. Local variations in radial thickness of the known Luer lock connectors, attributable to large dimensional variations between the thread ribs and groves along the axial length of the connector, further exacerbate variations in in molten plastic flow and subsequent cooling, during injection molding of the connectors. A particular location of concern with the Luer connector is uniformity of the tapered Luer engagement surface. Localized flow and cooling variations during the injection molding process may lead to higher rejection rates of finish molded Luer components that do not meet dimensional quality standards. Localized variations in Luer component wall thickness are more readily apparent in the following description of a known medical device, which incorporates an ISO compliant Luer connector.

FIG. 1 depicts a known syringe 20 with an ISO 80369-7 compliant syringe male Luer lock connector 22 and a known, mating syringe cap 24 with a similarly compliant female Luer lock connector. The known syringe cap 24 is shown in FIGS. 2-4 and is sold under the registered trademark POSIFLUSH®. The syringe cap 24 has a monolithic, molded-polymer, tubular body 26, with a distal end 28 and a proximal end 30. An annular grip surface 32 is incorporated on the distal end 28 and extends axially to a hub portion 33. The hub portion 33 transverses the interior of the tubular body 26, which isolates the grip surface 32 from a female Luer lock connector 34 that extends axially to the proximal end 30 of the tubular body 26. The annular grip surface 32 defines respective inner circumferential walls 36 and outer circumferential walls 38 circumferential walls and a local grip wall thickness to therebetween. Circumferentially oriented grip fins 40 extend radially and axially from the outer circumferential wall 38.

In the known syringe cap 24, the female Luer lock connector 34 defines respective inner circumferential walls 42 and outer circumferential walls 44 that define varying local wall thicknesses therebetween; two of which are shown as t_{C1} and t_{C2}. The outer circumferential wall 44 of the Luer lock connector 34 defines two-lead or two-starting thread, helical, external threads 46, which are shown as 46_{LT1} and 46_{LT2} in FIG. 4. The external threads 46 each comprise first 48 and second 50 flank portions, a bridging crest 52, and a root 54. The root 54 and first 48 and second 50 flank portions define a helical groove 56, while the crest 52 and the first and second flank portions define helical rib 58. The helical groove 56 and the helical rib 58 have trapezoidal, axial cross-section profiles. FIGS. 2 and 3 show various dimensions of the Luer connector 34. The major diameter D_{MAJ} corresponds to the helical rib height/thread depth 60 at the thread crest 52, and the minor diameter D_{MIN} corresponds to the diameter of the outer circumferential wall 44. Root width 62, crest width 64, thread angle 66, helix angle α, and thread pitch P₁ are denoted by dimensional arrows. Thread lead is defined as thread pitch P₁ multiplied by the number of thread starts (syringe cap 24 has two thread starts).

As shown in FIG. 3, the inner circumferential wall 42 of the Luer connector 34 forms a circumferential surface of a female Luer mating cavity 68. The hub 33 forms a closed, distal axial end 70 of the female Luer mating cavity 68. An open proximal end 71 of the female Luer mating cavity 68 is in communication with the proximal end 30 of the tubular body 26. Axial length L of the cavity 68 is measured between its distal axial end 70 and its open axial end 71. The hub 33 includes a molding sprue 72 with a distal end 74 that projects toward the distal axial end 28 of the tubular body 26.

The syringe cap 24 is typically injection molded within a mold whose cavity conforms to the outer surface profile of the cap's tubular body 26. Molten plastic is introduced into the mold through the zone of the mold that forms the distal end 74 of the molding sprue 72 and spreads radially into the zone that forms the hub 33. Thereafter, molten plastic travels axially and radially along the annular-shaped portions of the mold that form the grip surface 32 and the female Luer lock 34. The radial thickness of the annular grip surface portion 32 of the cap 24 is relatively uniform and is less than the radial thickness of the corresponding Luer connector portion 34, with thickness measured between the latter's inner 42 and outer 44 circumferential surfaces plus additional thickness and material volume occupied by the ribs 58 of the two helical threads 46. The annular grip surface portion 32 of the cap 24 cools more rapidly than the corresponding Luer connector portion 34; hence additional cooling time is required for the Luer connector portion 34.

Table 1 specifies selected, typical axial, cross-sectional dimensions of the Luer connector portion 34 of the known syringe cap 24 in millimeters and angular degrees. Manufacturing tolerances for these specified dimensions of such known syringe caps 24 vary by roughly +/−0.07 mm.

TABLE 1

| Helix angle α | 11.98 | |
|---|---|---|
| Thread angle 66 | 46 | |
| Thread depth/rib height 60 | 0.43 | |
| Root axial width | 0.53 | |
| Crest axial width | 1.67 | |
| Thread lead | 5.08 | |
| Thread pitch P₁ | 2.54 | |
| Axial cross-sectional area of | 0.79 | mm² (approximate) |
| helical rib 58 | | |
| Average local wall thickness | 1.3 | (approximate) |
| t_{C2} at helical groove 56 | | |
| Average local wall thickness | 1.73 | (approximate) |
| t_{C1} at helical rib 58 | | |
| Aggregate, axial cross- | 3.55 | mm² (approximate) |
| sectional area of helical ribs 58 | | |
| (approximately 5 ribs) (R) | | |
| Aggregate, axial cross- | 21.3 | mm² (approximate) |
| sectional area of tubular wall of | | |
| the Luer connector 34 (W) | | |
| Percentage ratio of Aggregate, | 16.6% | (approximate) |
| axial cross-sectional areas of | | |
| helical ribs 58 and the tubular | | |
| wall of Luer connector 34 | | |
| (R)/(W) | | |

The relatively large axial, cross-sectional area of each helical rib 58 is approximately 0.79 mm² and the aggregate axial, cross-sectional area of all approximately five helical ribs represent approximately 16.6 percent of the comparable aggregate axial, cross-sectional area of the entire tubular wall of the Luer connector 34. Varying tubular and helical rib thickness within the Luer connector 34 increases risk of discontinuities and uneven surface profile of the female Luer mating cavity 68 during injection molding of the syringe cap 24.

### SUMMARY

Medical devices incorporate four-lead thread, Luer connectors, also referred to as four-start thread, Luer connectors. Axial, cross-sectional area of these four-start/lead thread Luer connectors is significantly smaller than that of known, ISO80369-7 compliant Luer connectors. A Luer connector of the present disclosure comprises a monolithic, molded-polymer, tubular body having inner and outer wall circumferential surfaces and four-lead helical threads formed on one of the wall circumferential surfaces. The helical threads circumscribe a nested, concentrically aligned, frusto-conical, Luer-tapered, mating surface. The four-lead thread configuration reduces axial, cross-sectional area of each thread, which facilitates more uniform mold filling and cooling while injection molding the medical device, more uniform finished product, especially along the mating Luer surface of the connector, and material savings, compared with traditional two-lead thread Luer connectors. Some medical device embodiments of the present disclosure incorporate four-lead thread, female Luer connectors, such as syringe caps and intravenous catheters. Other embodiments of the present disclosure incorporate male Luer connectors, such as syringes.

The lead thread, with four starts, significantly reduces cross-sectional area of each individual helical thread and also reduces the overall average wall thickness of the Luer connector, which facilitates more consistent molding quality of the injection molded parts. The thinner cross-sectional threads provide excellent fluid sealing between mated male and female Luer connectors, as there are twice as many abutting sealing surfaces present in the Luer connectors of the present disclosure, as compared to known, two start helical thread, Luer connectors.

One aspect of the present disclosure pertains to a Luer connector comprising a monolithic, molded-polymer, tubular body having inner and outer wall circumferential surfaces and four-lead helical threads formed on one of the wall circumferential surfaces, the helical threads circumscribing a nested, concentrically aligned, frusto-conical, Luer-tapered, mating surface.

Another aspect of the present disclosure pertains to a component for a vascular access system, which includes a monolithic, molded-polymer body forming a female Luer connector defining an axial centerline. The connector has inner and outer wall circumferential surfaces, concentric with the axial centerline, which define a connector wall thickness therebetween relative to the axial centerline. The connector has four-lead, male, external helical threads on the outer wall circumferential surface. Each helical thread has a respective, local, axial cross-section about the outer wall. The helical threads circumscribe a nested, concentrically aligned, frusto-conical, Luer-tapered, female mating surface that is formed in the inner wall circumferential surface. In some embodiments, aggregate local, cross-sectional area of all threads aligned along any axial cross-section along the axial centerline of the connector is less than twelve percent (12%) of that of corresponding local, axial cross-sectional area of the connector wall. One or more aspects include a patient vascular port.

Yet another aspect of the present disclosure pertains to a syringe cap incorporating a monolithic, molded-polymer, tubular body, which defines an axial centerline. The tubular body incorporates an annular grip surface on a distal axial end and a female Luer connector on a proximal end. Relative to the axial centerline of the tubular body, respective inner and outer wall circumferential surfaces of the grip surface define a grip wall thickness, and respective inner and outer wall circumferential surfaces of the female Luer connector define a connector wall thickness therebetween. The outer wall circumferential surface of the female Luer connector defines four-lead, male, external helical threads. Each helical thread has a respective, local, axial cross-section about the outer wall, A frusto-conical, Luer-tapered, female mating surface is formed in the inner wall circumferential surface of the female Luer connector, in nested, coaxial alignment with the helical threads. Aggregate local, cross-sectional area of all threads aligned along any axial cross-section along the axial centerline of the connector is less than twelve percent (12%) of that of corresponding local, axial cross-sectional area of the connector wall.

### BRIEF DESCRIPTION OF THE DRAWINGS

Exemplary embodiments of the disclosure are further described in the following detailed description in conjunction with the accompanying drawings, in which:

FIG. 1 is an isometric view of an embodiment of a prior art medical syringe and prior art syringe cap, each with mating, two-lead thread, respective male and female Luer connectors;

FIG. 2 is an elevational view of the prior art syringe cap of FIG. 2;

FIG. 3 is an axial, cross-sectional view of the prior art syringe cap of FIG. 2;

FIG. 4 is an isometric view of the Luer connector of the prior art syringe cap of FIG. 2, showing its two-lead, helical threads;

FIG. 5 is an elevational view of a syringe cap, which incorporates the four-lead helical thread, Luer connector of the present disclosure;

FIG. 6 is an axial, cross-sectional view of the syringe cap of FIG. 5;

FIG. 7 is an isometric view of the Luer connector of the syringe cap of FIG. 5, showing its four-lead, helical threads;

FIG. 8 is a partial, axial, cross-sectional view of a syringe with a male Luer lock, which incorporates the four-lead helical thread, Luer connector of the present disclosure; and

FIG. 9 is an isometric view of an intravenous catheter, which incorporates a four-lead helical thread, female Luer connector of the present disclosure.

To facilitate understanding, identical reference numerals have been used, where possible, to designate identical elements that are common to the figures. The figures are not drawn to scale.

### DETAILED DESCRIPTION

Medical devices incorporate four-lead thread, Luer connectors, also referred to as four-start thread, Luer connectors. The Luer connector comprises a monolithic, molded-polymer, tubular body having inner and outer wall circumferential surfaces and four-lead helical threads formed on one of the wall circumferential surfaces. The helical threads circumscribe a nested, concentrically aligned, frusto-conical, Luer-tapered, mating surface. The four-lead thread configuration reduces axial, cross-sectional area of each thread, which facilitates more uniform mold filling and cooling while injection molding the medical device, more uniform finished product and material savings, compared with traditional two-lead thread Luer connectors. Some embodiments of medical devices incorporate four-lead thread, male Luer connectors, such as syringes. Other embodiments of medical devices incorporate four-lead thread, female Luer connectors, such as syringe caps and intravenous catheters, and hubs.

In this disclosure, where generally applicable, a convention is generally followed wherein the distal end of the device is the end closest to a patient, and the proximal end of the device is the end away from the patient and closest to a clinician or other medical practitioner. However, for better contextual understanding in this disclosure, an outwardly facing axial end of a Luer connector, which is intended to mate with a complementary Luer connector (e.g., male to female or female to male) is sometimes explicitly referred to as a “distal end of a Luer connector”. As used herein, the use of “a,” “an,” and “the” includes the singular and plural.

As previously described herein, the term “Luer connector” refers to a connection collar that is the standard way of attaching syringes, catheters, hubbed needles, IV tubes, etc. to each other. As also previously described herein, ISO 80369-7:2016 defines a specification for standard Luer connectors. The phrases “male Luer connector”, “female Luer connector”, male standard Luer connector”, “female standard Luer connector”, “male Luer lock connector” and “female Luer lock connector” shall refer to connectors having the dimensions described in ISO 80369-7, which is hereby incorporated by reference in its entirety.

As would be readily appreciated by skilled artisans in the relevant art, while descriptive terms such as “tip”, “hub”, “thread”, “protrusion/insert”, “tab”, “slope”, “wall”, “top”, “side”, “bottom” and others are used throughout this specification, as applicable, to facilitate understanding, it is not intended to limit any components that can be used in combinations or individually or to require specific spatial orientations, to implement various aspects of the embodiments of the present disclosure.

Before describing several exemplary embodiments of the disclosure, it is to be understood that the disclosure is not limited to the details of construction or process steps set forth in the following description. The disclosure is capable of other embodiments and of being practiced or being conducted in many ways. For example, where the Luer couplings disclosed herein have first and second mating fitting portions shown in one configuration (e.g., mating male and female connections), in other embodiments, the location and orientation of the mating fitting portions are reversed.

The matters exemplified in this description are provided to assist in a comprehensive understanding of exemplary embodiments of the disclosure. Accordingly, those of ordinary skill in the art will recognize that various changes and modifications of the embodiments described herein can be made without departing from the scope and spirit of the disclosure. Also, descriptions of well-known functions and constructions are omitted for clarity and conciseness.

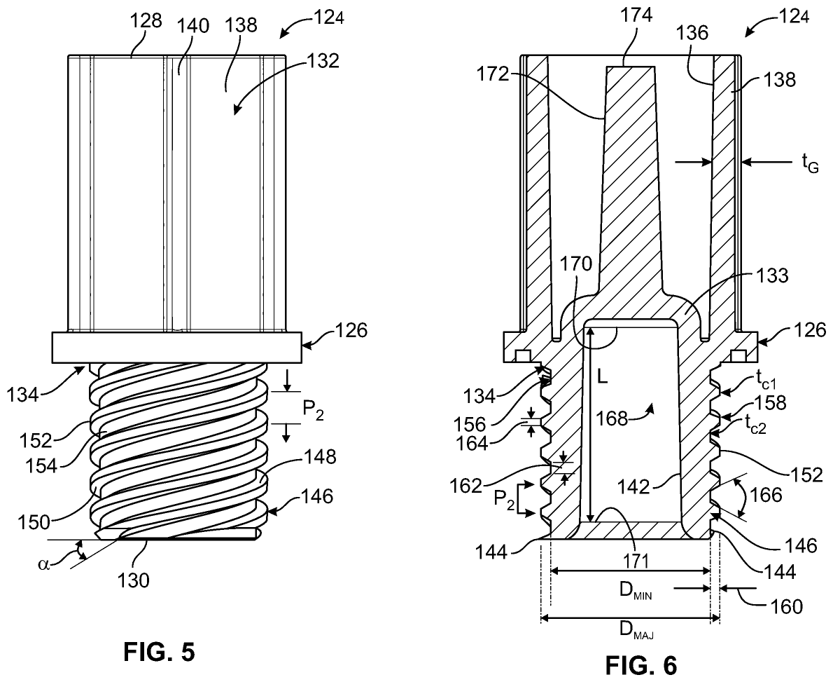

Referring generally to FIGS. 5-7, a first embodiment of a Luer connector is a female Luer lock connector that is incorporated within a syringe cap 124. The syringe cap 124 has a monolithic, molded-polymer, tubular body 126, with a distal end 128 and a proximal end 130. An annular grip surface 132 is incorporated on the distal end 128 and extends axially to a hub portion 133. The hub portion 133 transverses the interior of the tubular body 126, which isolates the grip surface 132 from a female Luer lock connector 134 that extends axially to the proximal end 130 of the tubular body 126. The annular grip surface 132 defines respective inner 136 and outer 138 circumferential walls and a local grip wall thickness to therebetween. Circumferentially oriented grip fins 140 extend radially and axially from the outer circumferential wall 138.

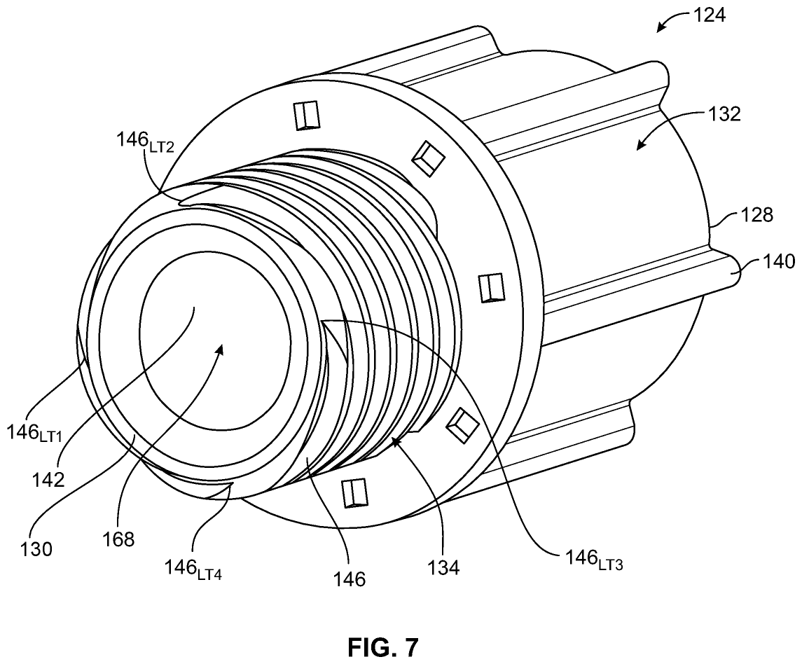

In the syringe cap 124, the female Luer lock connector 134 defines respective inner 142 and outer 144 circumferential walls define varying local wall thicknesses therebetween; two of which are shown as t_{C1} and t_{C2}. The outer circumferential wall 144 of the Luer lock connector 134 defines four-lead or four-starting thread, helical, external threads 146, which are shown as 146_{LT1}, 146_{LT2}, 146_{LT3}, and 146_{LT4} in FIG. 7. The four external threads 146 start at the proximal axial end 130 of the syringe cap 124 and are circumferentially clocked at 90-degree angles. Four start threads, clocked at 90-degrees engage easier with a mating, male Luer lock component as compared to two-start thread Luer Locks. Each of the respective helical threads 146 comprise first 148 and second 150 flank portions, a bridging crest 152, and a root 154. The root 154 and first 148 and second 150 flank portions define a helical groove 156, while the crest 152 and the first and second flank portions define helical rib 158. The helical groove 156 and the helical rib 158 have trapezoidal, axial cross-section profiles. FIGS. 5 and 6 show various dimensions of the Luer connector 134. The major diameter D_{MAJ} corresponds to the helical rib height/thread depth 160 at the thread crest 152, and the minor diameter D_{MIN} corresponds to the diameter of the outer circumferential wall 144. Root width 162, crest width 164, thread angle 166, helix angle α, and thread pitch P₂ are denoted by dimensional arrows. Thread lead is defined as thread pitch P₂ multiplied by the number of thread starts (syringe cap 224 has four thread starts).

As shown in FIG. 6, the inner circumferential wall 142 of the Luer connector 134 forms a circumferential surface of a female Luer mating cavity 168. The hub 133 forms a closed, distal axial end 170 of the female Luer mating cavity 168. An open proximal end 171 of the female Luer mating cavity 168 is in communication with the proximal end 130 of the tubular body 126. Axial length L of the cavity 168 is measured between its distal axial end 170 and its open axial end 171. The hub 133 includes a molding sprue 172 with a distal end 174 that projects toward the distal axial end 128 of the tubular body 126.

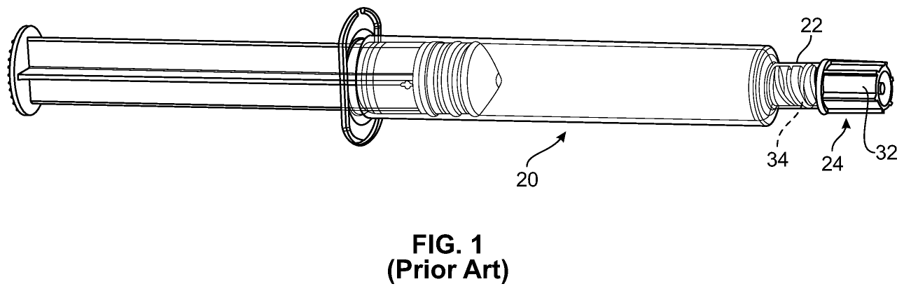

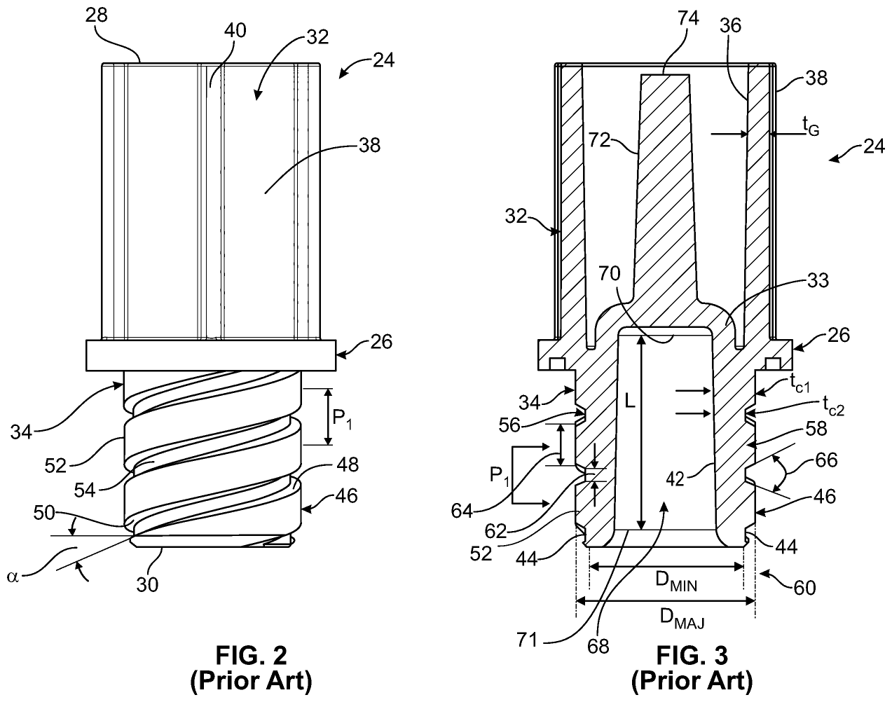

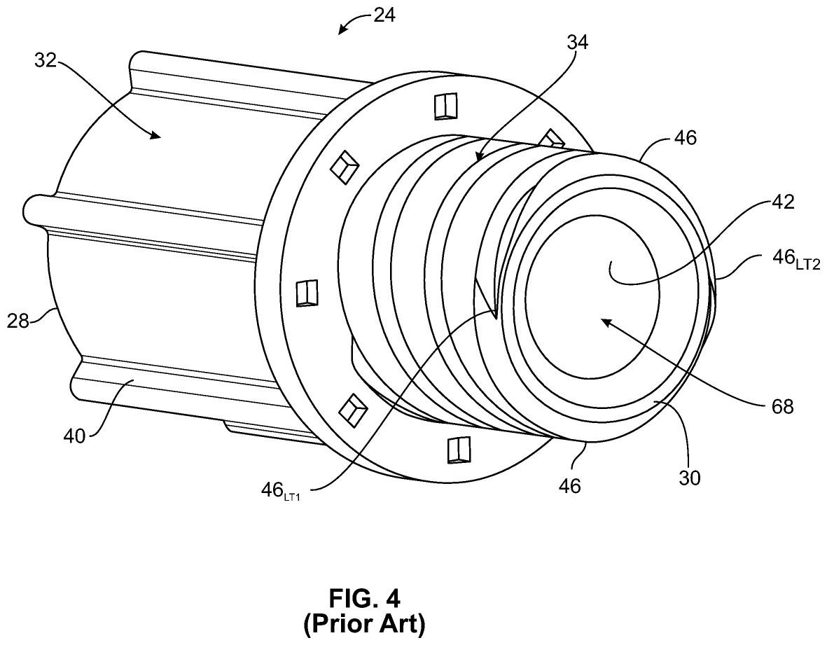

The syringe cap 124 is typically injection molded within a mold whose cavity conforms to the outer surface profile of the cap's tubular body 126. Molten plastic is introduced into the mold through the zone of the mold that forms the distal end 174 of the molding sprue 172 and spreads radially into the zone that forms the hub 33. Thereafter, molten plastic travels axially and radially along the annular-shaped portions of the mold that form the grip surface 132 and the female Luer lock 134. The radial thickness of the annular grip surface portion 132 of the cap 24 is relatively uniform and is less than the radial thickness of the corresponding Luer connector portion 134, between the latter's inner 142 and outer 144 circumferential surfaces plus additional volume occupied by the ribs 158 of the four helical threads 146. However, as described below, the thread pitch P₂ for the four start threads is one-half that of the comparable two thread start cap 24 of FIG. 3, while both cap designs have a common thread lead dimension. This results in significantly smaller axial, cross-sectional area of each thread rib 158 as compared to that of the known two-thread start cap 24. While the annular grip surface portion 132 of the cap 124 cools more rapidly than the corresponding Luer connector portion 134 the smaller axial, cross-sectional area of each thread rib 158 reduces overall cooling time and increases mold filling efficiency as compared to those of the Luer connector portion 34 of the known syringe cap 24 of FIGS. 1-4.

When the syringe cap 124 is coupled to a corresponding syringe male Luer lock, the first thread flank portions 148 and second thread flank portions 150 of the cap abut in sealing engagement against corresponding flank portions of the male Luer lock, which prevents fluid leakage out of the syringe. Doubling the thread pitch P₂ of the four-thread start, syringe cap 124 doubles the number of first 148 and second 150 thread flank portions 148 of the cap 124 compared to the known cap 24, further reducing likelihood of fluid leakage out of the syringe.

Table 2 specifies local dimension including selected, typical axial, cross-sectional dimensions of the Luer connector portion 134 of the syringe cap 124 in millimeters and angular degrees. Manufacturing tolerances for these specified dimensions vary by roughly +/−0.07 mm. Thread pitch P₂ thread crest width 164 of the syringe cap 124 differ from those of the known syringe cap 24, but the remaining dimensions are identical, and in conformity with the existing ISO 80369-7:2016 standard.

TABLE 2

| Helix angle α | 11.98 | |
|---|---|---|
| Thread angle 166 | 46 | |
| Thread depth/rib height 160 | 0.43 | |
| Root axial width 162 | 0.53 | |
| Crest axial width 164 | 0.37 | |
| Thread lead | 5.08 | |
| Thread pitch P₂ | 1.27 | |
| Axial cross-sectional area of | 0.24 | mm²(approximate) |
| helical rib 158 | | |
| Average local wall thickness | 1.3 | (approximate) |
| t_{C2} at helical groove 156 | | |
| Average local wall thickness | 1.73 | (approximate) |
| t_{C1} at helical rib 158 | | |
| Aggregate, axial cross- | 2.4 | mm²(approximate) |
| sectional area of helical ribs | | |
| 158 (approximately 10 ribs) | | |
| (R) | | |
| Aggregate, axial cross- | 21.3 | mm²(approximate) |
| sectional area of tubular wall of | | |
| the Luer connector 134 (W) | | |
| Percentage ratio of Aggregate, | 11% | (approximate) |
| axial cross-sectional areas of | | |
| helical ribs 158 and the tubular | | |
| wall of Luer connector 134 | | |
| (R)/(W) | | |

As compared to the dimensions of the known, two-start thread, syringe cap 24, the relatively smaller axial, cross-sectional area of each helical rib 158 of the four-start thread, syringe cap 124 of the present disclosure is approximately 0.24 mm². The ratio (R)/(W) of the aggregate axial, cross-sectional area of all approximately ten helical ribs 158 (R) and the comparable aggregate axial, cross-sectional area of its entire tubular wall (W) of the Luer connector 134 is approximately 11 percent. The comparable dimensions of the syringe cap 24 are approximately 0.79 mm² axial, cross sectional area and 16.6 percent aggregate axial, cross-sectional area of its entire tubular wall. Therefore, the four-start thread, syringe cap 124 of the present disclosure reduces axial, cross-sectional area of each helical rib 158 by approximately 66 percent, compared to that of the helical rib 58 of the known two-start syringe cap 24. The syringe cap 124 also reduces the (R)/(W) ratio of aggregate axial, cross-sectional areas of the helical ribs 158 to that of the entire tubular wall of the Luer connector 134 to 11 percent, as compared to the 16.6 percent (R)/(W) ratio of the known syringe cap 24: a material-volume, ratio reduction of approximately 33 percent.

In computational mold flow analysis comparisons of the known syringe cap 24 and the syringe cap 124 of the present disclosure, during injection molding simulations, the cap 124 exhibited more uniform local temperature of the injected plastic and cooled quicker than the known cap 24. Based on past manufacturing experience, greater uniformity of local temperature within a mold an quicker cooling times increases overall uniform quality of injection molded components and less rejection of components for failure to meet manufacturing specifications. Reduction in axial, cross-sectional area of each helical rib 158 of the syringe cap 124 of the present disclosure also reduces the volume of plastic material needed to fabricate the part, which results in lower manufacturing cost, and lower environmental impact associated with production and disposal of used plastic caps.

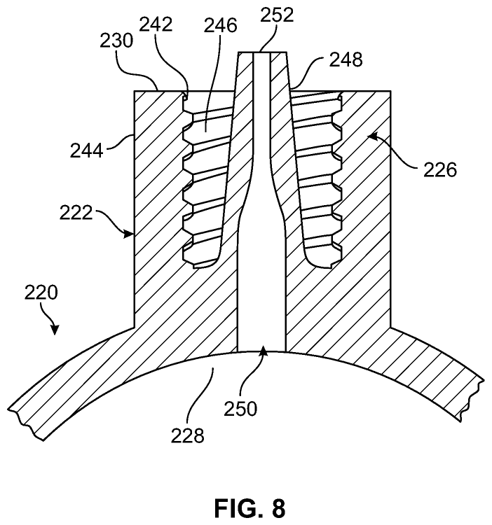

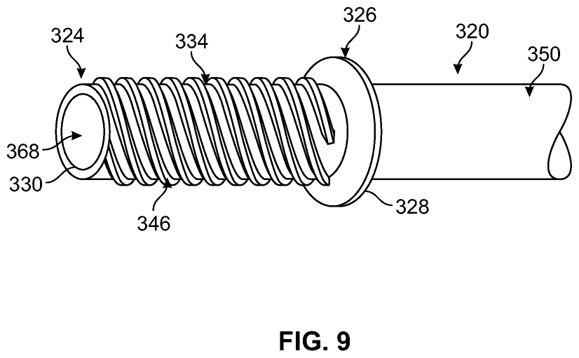

FIG. 8 shows a male Luer lock embodiment of Luer connector of the present disclosure incorporated within syringe 220. The male Luer lock 222 has a monolithic, molded-polymer, tubular body 226 with a proximal axial end 228, a distal axial end 230, an inner circumferential wall 242, and an outer circumferential wall 244. Four-start, internal helical threads 246 are formed in the inner circumferential wall 242; they circumscribe a tubular, male Luer mating surface 248. The syringe lumen 250 and syringe outlet 252 are formed within the tubular, male Luer mating surface 248.

FIG. 9 shows an intravenous (IV) catheter 320 that incorporates a female, Luer lock connector 324 of the present disclosure. The Luer connector 324 has a tubular body 326, a proximal end 328 of which incorporates a female Luer lock cavity 368. The female Luer lock cavity 368 is circumscribed by four-start helical threads 346, which define ribs and grooves as described with respect to the syringe cap 134. A distal axial end 330 of the tubular body 326 is coupled to a flexible cannula 350. Details of the cannula needle are not shown.

In various embodiments, the cap 124, the syringe male Luer lock 222, the IV catheter 320 and other medical devices comprising a four-start Luer connector of the present disclosure is constructed from any of a number of types of plastic materials such as polycarbonate, polypropylene, polyethylene, glycol-modified polyethylene terephthalate, acrylonitrile butadiene styrene or any other injection-moldable, medical-grade, plastic material used in medical devices.

Various aspects of the present disclosure include a patient vascular port.

Reference throughout this specification to “one embodiment,” “certain embodiments,” “various embodiments,” “one or more embodiments” or “an embodiment” means that a particular feature, structure, material, or characteristic described in connection with the embodiment is included in at least one embodiment of the disclosure. Thus, the appearances of the phrases such as “in one or more embodiments,” “in certain embodiments,” “in various embodiments,” “in one embodiment” or “in an embodiment” in various places throughout this specification are not necessarily referring to the same embodiment of the disclosure. Furthermore, the particular features, structures, materials, or characteristics may be combined in any suitable manner in one or more embodiments.

Although the disclosure herein provided a description with reference to particular embodiments, it is to be understood that these embodiments are merely illustrative of the principles and applications of the disclosure. It will be apparent to those skilled in the art that various modifications and variations can be made to the present disclosure without departing from the spirit and scope thereof. Thus, it is intended that the present disclosure include modifications and variations that are within the scope of the appended claims and their equivalents.

What is claimed is:

1. A Luer connector comprising a monolithic, molded-polymer, tubular body having inner and outer wall circumferential surfaces and four-lead helical threads formed on one of the wall circumferential surfaces, the helical threads circumscribing a nested, concentrically aligned, frusto-conical, Luer-tapered, mating surface.

2. The Luer connector of claim 1, further comprising a female Luer connector, the four-lead helical threads formed on the outer wall circumferential surface and the tubular body, circumscribing a nested, concentrically aligned, frusto-conical, Luer-tapered, female mating surface that is formed in the inner wall circumferential surface.

3. A syringe cap, which incorporates the Luer connector of claim 2.

4. An intravenous catheter, which incorporates the Luer connector of claim 2.

5. The Luer connector of claim 1, further comprising a male Luer connector, the four-lead helical threads formed on the inner wall circumferential surface of the tubular body, circumscribing a nested, concentrically aligned, frusto-conical, Luer-tapered, male mating surface that is formed on a first axial end of the tubular body.

6. A medical syringe, which incorporates the Luer connector of claim 5.

7. A component for a vascular access system, comprising a monolithic, molded-polymer body forming a female Luer connector defining an axial centerline, the connector having inner and outer wall circumferential surfaces, concentric with the axial centerline, which define a connector wall thickness therebetween relative to the axial centerline; four-lead, male, external helical threads on the outer wall circumferential surface, each helical thread having a respective, local, axial cross-section about the outer wall, the helical threads circumscribing a nested, concentrically aligned, frusto-conical, Luer-tapered, female mating surface that is formed in the inner wall circumferential surface; and wherein aggregate local, cross-sectional area of all threads aligned along any axial cross-section along the axial centerline of the connector is less than twelve percent (12%) of that of corresponding local, axial cross-sectional area of the connector wall.

8. The component of claim 7, further comprising a syringe cap.

9. The component of claim 8, further comprising each of the four-lead, male external helical threads having the following local dimensions relative to the axial centerline (in millimeters and degrees):
an 11.98-degree helix angle;
a 46-degree thread angle;
a 0.43 thread depth;
a 0.53 root axial width;
a 0.37 crest axial width;
a 5.08 thread lead; and
a 1.27 thread pitch.

10. The component of claim 7, further comprising a patient vascular port.

11. A syringe cap comprising:
a monolithic, molded-polymer, tubular body, defining an axial centerline, the tubular body incorporating an annular grip surface on a distal axial end thereof and a female Luer connector on a proximal end of the tubular body;
respective inner and outer wall circumferential surfaces of the annular grip surface defining a grip wall thickness therebetween relative to the axial centerline, and respective inner and outer wall circumferential surfaces of the female Luer connector defining a connector wall thickness therebetween relative to the axial centerline;
four-lead, male, external helical threads on the outer wall circumferential surface of the female Luer connector each helical thread having a respective, local, axial cross-section about the outer wall; and
a frusto-conical, Luer-tapered, female mating surface, formed in the inner wall circumferential surface of the female Luer connector, in nested, coaxial alignment with the helical threads;
wherein aggregate local, cross-sectional area of all threads aligned along any axial cross-section along the axial centerline of the connector is less than twelve percent (12%) of that of corresponding local, axial cross-sectional area of the connector wall.

12. The syringe cap of claim 11, further comprising each of the four-lead, male external helical threads having the following local dimensions relative to the axial centerline (in millimeters and degrees):
an 11.98-degree helix angle;
a 46-degree thread angle;
a 0.43 thread depth;
a 0.53 root axial width;
a 0.37 crest axial width;
a 5.08 thread lead; and
a 1.27 thread pitch.
